(19) United States
(12) Patent Application Publication
(10) Pub. No.: US 2005/0075679 A1
(43) Pub. Date: Apr. 7, 2005

(54) Methods and apparatuses for treating neurological disorders by electrically stimulating cells implanted in the nervous system

(76) Inventors: Bradford E. Gliner, Sammamish, WA (US); Alan J. Levy, Bellevue, WA (US); Jeffrey Balzer, Allison Park, PA (US); Andrew D. Firlik, New Canaan, CT (US); W. Douglas Sheffield, Seattle, WA (US); Allen Wyler, Seattle, WA (US)

Correspondence Address: PERKINS COIE LLP;PATENT-SEA, P.O. BOX 1247, SEATTLE, WA, 98111-1247, US

(21) Appl. No.: 10/842,052

(22) Filed: May 7, 2004

Related U.S. Application Data
(63) Continuation-in-part of application No. 10/261,116, filed on Sep. 30, 2002.

(51) Int. Cl. A61N 1/18

(52) U.S. Cl. 607/45

(57) ABSTRACT

Methods and apparatuses for treating neurological disorders by electrically stimulating cells implanted in the nervous system are disclosed. A method in accordance with one aspect of the invention includes preparing cells for implantation while the cells are in a first, at least partially undifferentiated state. The cells are then implanted at an implantation site within the patient's skull cavity while in the first state, and at least one electrode is positioned to be in electrical communication with the implantation site. The patient's neural dysfunction is at least partially corrected by differentiating the cells at least until the cells achieve a second state, with the cells in the second state having an increased level of differentiation and increased neurocharacteristics when compared to the cells in the first state. Differentiating the cells can include applying an electrical potential to the at least one electrode while the electrode is in electrical communication with the implantation site. In further aspects of the invention, the cells are implanted directly into the tissue without being carried by an electrically conductive substrate, and/or the electrode is removed from the patient without removing the implanted cells, for example, after stimulation has been completed.

### CROSS-REFERENCE TO RELATED APPLICATION

The present application is a continuation-in-part of pending U.S. application Ser. No. 10/261,116 filed Sep. 30, 2002 and incorporated herein in its entirety by reference.

### TECHNICAL FIELD

The present invention is directed generally toward methods and apparatuses for treating neurological disorders by electrically stimulating cells implanted in the nervous system.

### BACKGROUND

A wide variety of mental and physical processes are controlled or influenced by neural activity in particular regions of the brain. For example, various physical or cognitive functions are directed or affected by neural activity within the various regions of the cerebral cortex. For most individuals, particular areas of the brain appear to have distinct functions. In most people, for example, the areas of the occipital lobes relate to vision; the regions of the left inferior frontal lobes relate to language; portions of the cerebral cortex appear to be involved with conscious awareness, memory, and intellect; and particular regions of the cerebral cortex as well as the basal ganglia, the thalamus, and the motor cortex cooperatively interact to facilitate motor function control.

Many problems or abnormalities with body functions can be caused by damage, disease and/or disorders of the nervous system, which includes the brain, the spinal cord and peripheral nerves. A stroke, for example, is one very common condition that damages the brain. Strokes are generally caused by emboli (e.g., vascular obstructions), hemorrhages (e.g., vascular ruptures) or thrombi (e.g., vascular clots) in a specific region of the cortex, which in turn generally causes a loss or impairment of a neural function (e.g., neural functions related to face muscles, limbs, speech, etc.). Stroke patients are typically treated using physical therapy to rehabilitate the loss of function of a limb or other affected body part. For most patients, little can be done to improve the function of the affected limb beyond the recovery that occurs naturally without intervention.

One existing physical therapy technique for treating stroke patients constrains or restrains the use of a working body part of the patient to force the patient to use the affected body part. For example, the loss of use of a limb is treated by restraining the other limb. Although this type of physical therapy has shown some experimental efficacy, it is expensive, time-consuming and little-used. Stroke patients can also be treated using physical therapy plus adjunctive therapies. For example, some types of drugs, such as amphetamines, that increase the activation of neurons in general, appear to enhance neural networks; these drugs however, have limited efficacy because they are very non-selective in their mechanisms of action and cannot be delivered in high concentrations directly to the site where they are needed. Therefore, there is a need to develop effective treatments for rehabilitating stroke patients and patients having other types of neurological disorders. Such disorders include Alzheimer's disease, Parkinson's disease, Hodgkins disease, Huntington's disease, essential tremor motion, and language disorders, such as aphasias.

Two additional approaches for addressing the loss of neural functionality include electrical stimulation of nerve cells and replacement of nerve cells. These two approaches have also been combined. For example, U.S. Pat. No. 6,095,148 to Shastri et al. (“Shastri”) discloses a method for electrically stimulating nerve cells prior to or after implantation in the body, using electrically conductive polymers as a substrate. This technique may be unnecessarily invasive. For example, it may be difficult to remove the substrate from the patient after the cells have been regenerated, differentiated, or altered with the electrically conductive polymer. Furthermore, the replacement cells may not grow in the desired directions to complete functional connections with other cells.

### BRIEF DESCRIPTION OF THE DRAWINGS

FIG. 1A is a flow diagram illustrating a method for treating neurological disorders in accordance with an embodiment of the invention.

FIGS. 1B and 1C illustrate a method for implanting cells and an electrical stimulator in accordance with an embodiment of the invention.

FIG. 2 is a schematic illustration of an implanted electrical stimulator positioned to apply an electrical current to implanted cells in accordance with an embodiment of the invention.

FIG. 3 is a partially schematic, approximately horizontal cross-sectional cut through a human brain illustrating sites for implanting and stimulating cells in accordance with further embodiments of the invention.

FIG. 4 is a partially schematic illustration of a stimulation system positioned to stimulate language centers of the brain in accordance with an embodiment to the invention.

FIG. 5 is a partially schematic illustration of a stimulation system configured in accordance with another embodiment of the invention.

FIG. 6 is a partially schematic, cross-sectional illustration of a stimulation system configured in accordance with another embodiment of the invention and implanted in a patient's skull.

FIG. 7 is a partially schematic cross-sectional illustration of a stimulation system having a driving member in accordance with another embodiment of the invention.

FIG. 8 is a partially schematic cross-sectional illustration of a stimulation system supported in accordance with another embodiment of the invention.

FIG. 9 is a partially schematic illustration of an arrangement for stimulating neural cells external to the patient in accordance with an embodiment to the invention.

FIGS. 10-11 illustrate methods for implanting and stimulating neural cells in accordance with further embodiments of the invention.

### DETAILED DESCRIPTION

The following disclosure describes several methods for treating neurological disorders and/or dysfunctions by implanting and/or electrically stimulating cells that have and/or develop neural characteristics. For example, methods in accordance with some embodiments of the invention include preparing at least partially undifferentiated cells for implantation, implanting the cells within a patient's skull cavity, and differentiating the cells to have increased neural characteristics by applying an electrical potential to at least one electrode in electrical communication with the cells. The cells can be implanted at a variety of locations, depending on the patient's disorder. For example, when the patient has suffered a stroke, the cells can be implanted at an infarct or peri-infarct region of the brain. If the patient suffers from movement disorders, the cells can be implanted at the motor cortex, basal ganglia, thalamus, or other centers of the brain responsible for controlling the patient's movements. If the patient suffers from language disorders, the cells can be implanted at or near the language centers of the brain.

In further embodiments, the process of increasing the neural characteristics and/or functionality of the cells can be enhanced by exposing the cells to growth factors, for example, IGF and/or GDNF. In other embodiments, aspects of the manner in which the electrical stimulation is applied to the cells can be controlled to enhance the neuronal development of the cells. Such characteristics include the voltage of the electrical stimulation, the current of the electrical stimulation, the pulse width, the pulse pattern, and/or the frequency or frequencies at which the electrical stimulation is varied.

In still further embodiments, the electrical stimulation can be used to alter characteristics of fully differentiated neural cells. For example, stimulation can be applied to fully differentiated, implanted cells to enhance connections between those cells and native cells of the patient. Such techniques can also be used to direct the growth of fully differentiated cells, for example, by directing an electrical current through the tissues surrounding the fully differentiated neural cells.

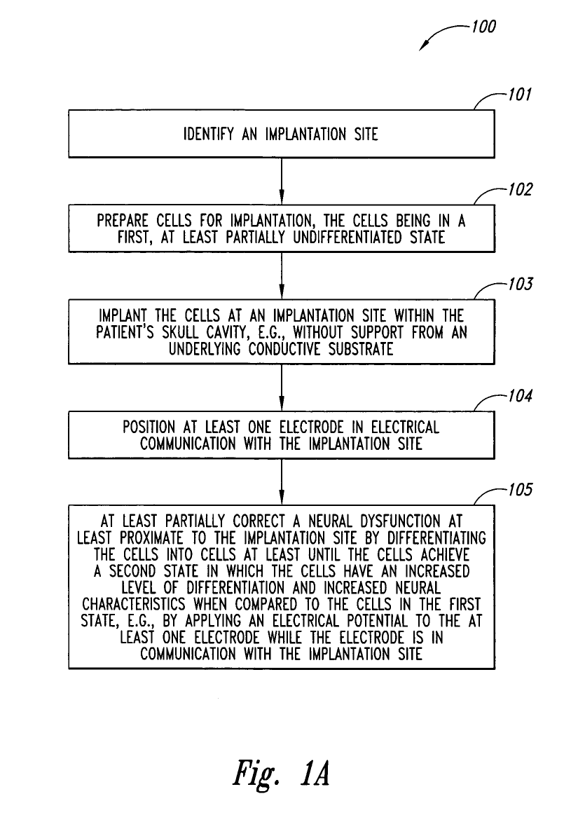

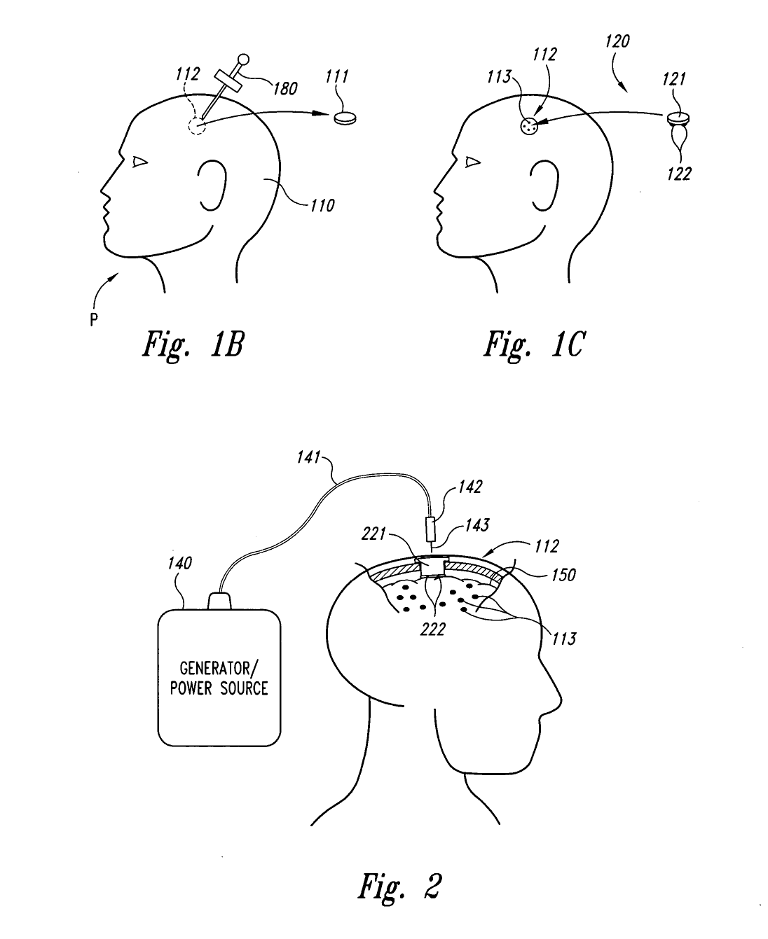

The specific details of certain embodiments of the invention are set forth in the following description and in FIGS. 1A-11. It will be appreciated that methods in accordance with other embodiments of the invention can include additional procedures or features different than those shown in FIGS. 1A-11. Accordingly, methods in accordance with several embodiments of the invention may not include all of the features shown in these Figures.

FIG. 1A is a flow chart illustrating a process for correcting a patient's neural dysfunction in accordance with an embodiment to the invention. The process 100 can include identifying an implantation site (process portion 101) and preparing cells for implantation, the cells being in a first, at least partially undifferentiated state (process portion 102). Accordingly, the cells in the first state can be completely undifferentiated, or can have achieved some incomplete level of differentiation. In either embodiment, the cells in the first state are capable of undergoing further differentiation.

The cells are then implanted at an implantation site within the patient's skull cavity while the cells are in the first state (process portion 103). In a particular aspect of this embodiment, the cells are implanted without support from a substrate. At least one electrode is positioned in electrical communication with the implantation site in process portion 104. In process portion 105, the neural dysfunction of and/or damage to the patient's nervous system at least proximate to the implantation site is at least partially corrected or reversed by differentiating the cells at least until the cells achieve a second state. In the second state, the cells have an increased level of differentiation and increased neural characteristics when compared to the cells in the first state. The correction or reversal is obtained at least in part by applying an electrical potential to the at least one electrode while the electrode is in electrical communication with the implantation site.

As used herein, the term “at least partially undifferentiated” when identifying a cell characteristic, includes a cell capable of differentiating or further differentiating from an initial state into a cell (such as a neuron) that exhibits increased signaling characteristics (e.g., increased electrical and/or chemical signaling characteristics) when compared to the cell in its initial state. The signaling characteristics can include action potential characteristics. An action potential occurs when a membrane potential of the cell (e.g., the resting membrane potential) surpasses a threshold level. When this threshold level is reached, and “all-or-nothing” action potential is generated. For example, once the threshold level is reached in a neuron, the neuron can “fire” an action potential, which propagates down the length of the axon of the neuron to cause the release of neurotransmitters from that neuron that will further influence adjacent neurons.

At least partially undifferentiated cells can include stem cells, progenitor cells, precursor cells, and cells having stem cell-like characteristics (e.g., blood cells that are modified to have such characteristics). Stem cells are characterized as being completely undifferentiated; they can divide without limit and when they divide each daughter cell can remain a stem cell or assume the physical and/or functional characteristics of a cell that it is replacing. For example, stem cells are capable of differentiating into neurons or glial cells. In one embodiment, a progenitor cell can be partially undifferentiated and can therefore have a more restricted potential cellular purpose than a stem cell. For example, some progenitor cells may only develop into neurons or glia. In one embodiment, a precursor cell can be even more differentiated than a progenitor cell and can have even more restricted cellular purposes. For example, a neuroblast can only become a neuron. In other embodiments, the at least partially undifferentiated cells can include other cell types. Accordingly, stem cells, progenitor cells, and precursor cells are representative examples, rather than an exhaustive list of at least partially undifferentiated cells.

FIGS. 1B-1C schematically illustrate a procedure for implanting cells in the brain in accordance with an embodiment of the invention. Referring first to FIG. 1B, the practitioner selects an implantation site 112 of a patient P at which the cells will be implanted. The practitioner can remove a skull section 111 from the patient's skull 110 directly over the implantation site 112. The practitioner can then implant the cells using a syringe 180 containing the cells suspended in a solution. In other embodiments, other techniques can be used to implant the cells at the implantation site 112. In any of these embodiments, the cells can be implanted at or near the surface of the brain and/or more deeply within the brain, depending on factors that include the overall condition of the patient's brain and/or the dysfunction that the implanted cells are to address.

Referring now to FIG. 1C, the practitioner can position a stimulation system 120 at least proximate to implanted cells 113 at the implantation site 112. The stimulation system 120 can accordingly be in electrical communication with the implantation site 113 and can provide electrical stimulation signals to a stimulation site that is at least proximate to the implantation site 113. In one aspect of this embodiment, the stimulation system 120 includes an implantable support 121 carrying one or more electrodes 122 (two are shown in FIG. 1B). The implantable support 121 can be positioned in the patient's skull 110 with the electrodes 122 at least proximate to the implantation site 112 and therefore the implanted cells 113. In one aspect of this embodiment, the electrodes 122 can be in direct contact with the implanted cells 113. In another embodiment, the electrodes 122 can communicate electrically with the implanted cells 113 via native cells positioned between the implanted cells 113 and the electrodes 122. The implantable support 121 can be positioned at the implantation site 112 before or after the implanted cells 113 are introduced at the implantation site 112. The implantable support 121 can also be removed from the patient (even if the electrodes 122 contact the implanted cells 113) without having a significant adverse impact on the implanted cells 113. Accordingly, when function of the stimulation system 120 has been completed (e.g., when the implanted cells 113 have developed to the point when electrical stimulation is no longer necessary), the support 121 can be removed with relative ease.

The growth, differentiation and/or development of the implanted cells 113 can be encouraged by other agents in addition to the electrical current described above. For example, the implanted cells 113 can be exposed to growth factors including IGF and/or GDNF, after implantation and/or before implantation. The growth factors can be introduced to the implanted cells 113 by existing techniques, for example, by virus transport, as disclosed by Lauerman in “Brain Work This Week,” v. 1, No. 30, incorporated herein in its entirety by reference.

One feature of an embodiment of the foregoing arrangement is that the implanted cells 113 can be introduced directly into the surrounding native tissue and stimulated either directly or via the surrounding tissue. Accordingly, this arrangement does not require an implanted substrate that supports the implanted cells and transmits electrical signals to the implanted cells. An advantage of this approach when compared with some conventional arrangements (such as that disclosed by Shastri in U.S. Pat. No. 6,095,148) is that it can reduce the complexity of the stimulation system 120 and the amount of non-native material that must be implanted in the patient P. For example, this approach does not require the implanted cells to be introduced into the brain on a conductive polymer substrate, which can be difficult if not impossible to remove from the patient's brain after the implanted cells 113 are fully developed and/or differentiated. Instead, electrical stimulation is provided by a device that can be removed from the brain with relative ease when it is no longer needed.

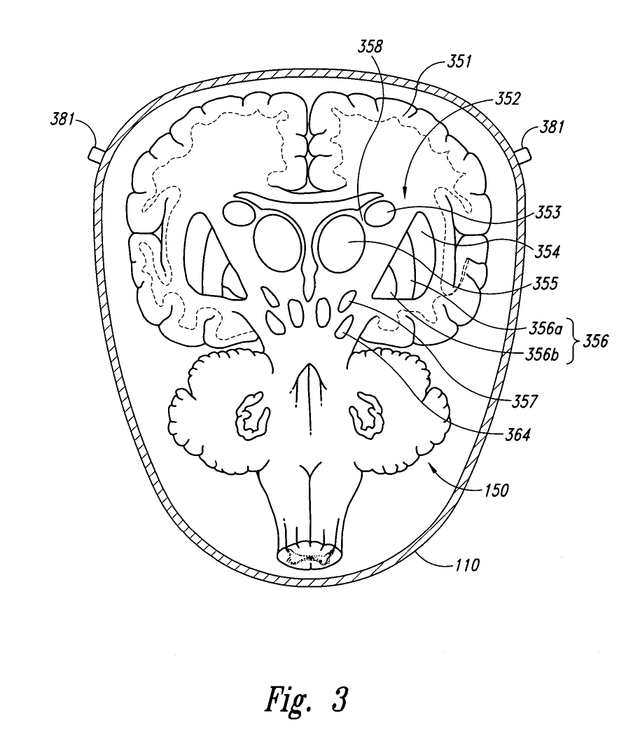

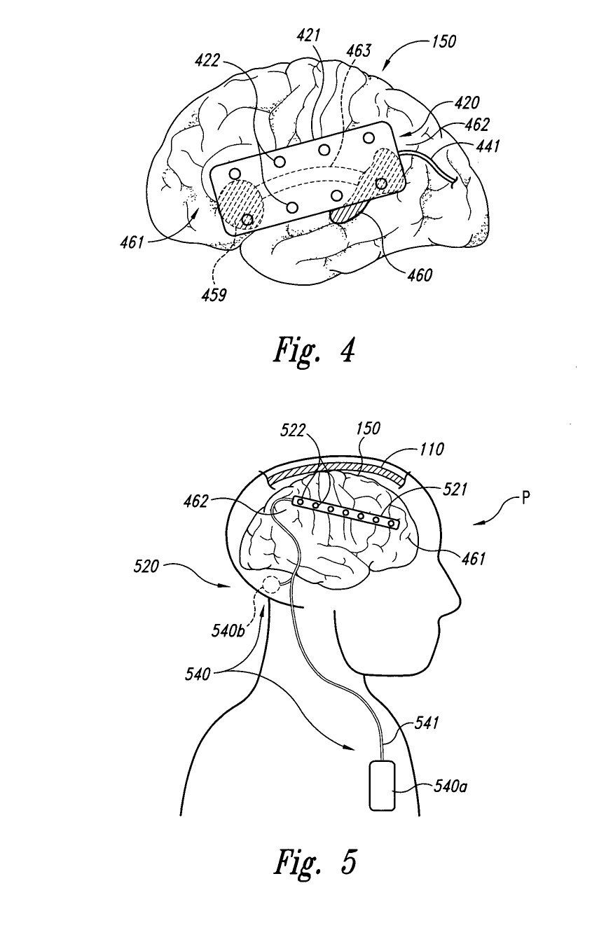

FIG. 2 schematically illustrates a procedure for electrically stimulating the cells 113 implanted in the patient's brain 150. In one aspect of this embodiment, an implantable support 221 includes a plurality of electrodes 222 positioned at least proximate to the implantation site 112. Accordingly, the electrodes 222 can direct an electrical current to the implanted cells 113 directly and/or via the native tissue surrounding the implanted cells 113. In one aspect of this embodiment, the electrical current can be delivered to the electrodes 222 proximate to the implanted cells 113 by a pulse generator 140 positioned external to the patient's body. Accordingly, the pulse generator 140 can be coupled to a lead 141 having a plug 142 and a needle 143 that, when inserted into the implantable support 221, provides reliable yet separable electrical communication with the electrodes 222. In other embodiments, the pulse generator 140 itself can be implanted in the patient P, as described in greater detail below with reference to FIGS. 4-8. In any of these embodiments, the implantation site 112 can have any of a number of locations relative to the brain 150, depending on the condition to be addressed by the procedure. Further details of representative implantation sites are described below with reference to FIGS. 3 and 4.

FIG. 3 illustrates an approximately horizontal section through the patient's brain 150, along with selected sites for cell implantation and electrical stimulation in accordance with embodiments of the invention. In some embodiments, the implantation and electrical stimulation techniques are directed toward addressing motor neuron dysfunctions. Accordingly, corresponding implantation and stimulation techniques can be directed toward those portions of the brain responsible for motor functions. These areas include the motor cortex 351, and/or other cortical areas, e.g., the supplementary motor area (SMA) and/or premotor areas. These areas also include the basal ganglia 352 and the thalamus 355. The basal ganglia 352 can in turn include the caudate nucleus 353, the putamen 354 and the globus pallidus 356. The globus pallidus 356 can in turn include a lateral segment 356a and a medial segment 356b. The thalamus 355 can include a ventralis intermedius nucleus 358. Other sites for implantation and stimulation that address motor functions include the subthalamic nucleus 357 and the substantia nigra 364. The specific locations of the foregoing sites can be more accurately determined with reference to fidicials 381 (fixed relative to the skull 110) or the patient's own anatomical landmarks which are visible on images of the brain 150. Further details of techniques for identifying target sites for implementation and electrical stimulation are described below.

One procedure for identifying an implantation site includes generating the intended neural activity remotely, and then detecting or sensing the location in the brain where the intended neural activity has been generated. The intended neural activity can be generated by applying an input that causes the signal to be sent to the brain. For example, in the case of a patient who has lost the use of a limb, the affected limb is moved and/or stimulated while the brain is scanned, using a known imaging technique that can detect neural activity. Such imaging techniques include functional magnetic resonance imaging (fMRI) techniques, magnetic resonance imaging (MRI) techniques, computed tomography (CT) techniques, single photon emission computed tomography (SPECT) techniques, positron emission tomography (PET) techniques and/or other techniques.

In one specific embodiment, the affected limb can be moved by the practitioner or the patient, stimulated by a sensory test (e.g., a pricking test), or subjected to peripheral electrical stimulation. The movement/stimulation of the affected limb produces a peripheral neural signal from the limb that is expected to generate a response neural activity in the brain. The location in the brain where this response neural activity is present can be identified using any of the foregoing imaging techniques. By peripherally generating the intended neural activity, this embodiment may accurately identify where the brain has recruited matter to perform the intended neural activity associated with the neural function. This location can be selected as a site for implanted cells.

Another method for identifying the implantation site includes identifying a location of the brain where the neural activity has changed in response to a change in the neural function of the patient. This embodiment does not necessarily require that the intended neural activity be generated by peripherally actuating or stimulating a body part. For example, the brain can be scanned for neural activity associated with the impaired neural function as the patient regains use of an affected limb or learns a task over a period of time. This embodiment, however, can also include peripherally generating the intended neural activity remotely from the brain, as explained above.

In still another embodiment, the implantation and stimulation site can be identified at a location of the brain where the intended neural activity is developing. This technique can be generally similar to other embodiments described above but can be used to identify stimulation site at (a) the normal region of the brain where the intended neural activity is expected to occur, in accordance with the functional organization of the brain and/or (b) a different region where the neural activity occurs because the brain is recruiting additional matter to perform the neural function. This particular embodiment includes monitoring neural activity at one or more locations where the neural activity occurs in response to the particular neural function of interest. For example, to enhance the ability to learn a particular task (e.g., playing a musical instrument, memorizing, etc.) the neural activity can be monitored while a person performs the task or thinks about performing the task. The implantation/stimulation sites can be defined by the areas of the brain where the neural activity has the highest intensity, the greatest increase, and/or other parameters that indicate areas of the brain that are being used to perform the particular task.

In other embodiments, similar techniques are used to identify areas of the brain for implantation and stimulation to correct other neural dysfunctions. For example, imaging techniques including MRI techniques can be used to locate infarct regions caused by strokes or other conditions. Cells can be implanted directly at the infarct regions, and/or at surrounding peri-infarct regions. In still further embodiments, the areas of the brain selected for implantation and stimulation are identified with reference to the patient's anatomical features.

In certain embodiments, a set of implantation and/or stimulation sites may be identified through an acquisition, measurement, generation, and/or analysis of electrophysiologically-based signals, such as coherence and/or partial coherence signals. Coherence may provide a measure of rhythmic or synchronous neural activity that may result from oscillatory signaling behavior associated with various neural pathways or loops. In general, coherence may be defined as a frequency-domain measure of synchronous activity and/or linear association between a first and a second signal. The first and second signals may be identical or different signal types. For example, depending upon embodiment details, a coherence measurement may be based upon two EMG signals; two EEG signals; two ECoG signals; two MEG signals; an EMG signal and an EEG, ECoG, or MEG signal; an EMG, EEG, ECoG, or MEG signal and a functional correlate signal (e.g., an accelerometer signal); or two functional correlate signals; or other signal type pairs. Particular manners of making and/or interpreting coherence measurements are described in detail in “Defective cortical drive to muscle in Parkinson's disease and its improvement with levodopa,” Stephan Salenius et al., Brain (2002), Vol. 125, p. 491-500; and “Intermuscular coherence in Parkinson's disease: relationship to bradykinesia,” Peter Brown et al., NeuroReport, Vol. 12, No. 11, Aug. 8, 2001. Those skilled in the art will understand that measurement or determination of coherence may involve multiple signal acquisitions, measurements, and/or recordings, potentially separated by quiescent intervals, and possibly mathematical procedures upon such signals, which may comprise for example, filtering, averaging, transform, statistical operations, and spectral analysis operations.

In yet further embodiments, generally similar techniques can be used to identify areas of the brain targeted for implantation and stimulation to correct language-based disorders, such as aphasias. FIG. 4 is a partially schematic, side view of the brain 150 illustrating the inferior parietal lobe 462 and the inferior frontal lobe 461. These regions typically house the language centers of the brain 150, including Broca's area 459, Wernicke's area 460, and the association fibers of the arcuate faciliculcus 463 extending therebetween.

To identify the particular portion of the brain to be targeted for implantation and stimulation, the practitioner can direct the patient to perform a language-based task that generates a neural response which can be made visible using any of the imaging techniques described above. In a particular aspect of this embodiment, the language-based task performed by the patient does not require the patient to actually vocalize. Instead, the patient can be directed to merely think of a word, letter, phrase or other language component. For example, the patient can be directed to silently generate a verb associated with a common noun, silently repeat a noun, silently retrieve a word based on a letter cue, or silently retrieve a word based on a visual cue. In any of these embodiments, the patient need not use motor neurons to execute the selected task. Accordingly, this technique can reduce or eliminate the recorded activity of motor neurons, which might otherwise clutter or obscure the cognitive, language-based information of interest. Further details of methods for obtaining such information are included in co-pending U.S. application Ser. No. ______ (Attorney Docket No. 33734.8055US01) entitled “Methods for Treating and/or Collecting Information Regarding Neurological Disorders, Including Language Disorders,” filed Dec. 10, 2003, and incorporated herein in its entirety by reference.

In some cases, only a site identified by the foregoing techniques is selected for implantation and stimulation. In other cases, a contralateral site (e.g., the corresponding site on the opposite hemisphere of the brain 150) is selected, in addition to or in lieu of the identified site. The selection of a particular site or contralateral site can depend upon the type of disorder treated and/or the overall condition of the patient's brain 150. In any of the foregoing embodiments, the practitioner can select a plurality of implantation and/or stimulation sites for a single patient. For example, the practitioner can select multiple sites if it is initially unclear which site will provide a benefit (and/or the greatest benefit) to the patient, and/or if it is determined that implantation and/or stimulation at a plurality of sites provides a greater benefit than implantation and/or stimulation at a single site.

Once the appropriate information regarding the patient's neural activity (or lack of neural activity) has been collected, the at least partially undifferentiated cells can be implanted in the brain 150 in a manner generally similar to that described above. In some embodiments, the implantation and stimulation may take place at more than one location of the brain 150. Accordingly, a stimulation system 420 having an elongated support 421 with multiple electrodes 422 can be positioned in the brain 150 to stimulate a variety of locations. An advantage of this arrangement is that a practitioner can stimulate multiple sites of the brain 150 (either simultaneously or sequentially) with a single system 420. In one embodiment, the practitioner can stimulate multiple sites (rather than a single site) to produce enhanced benefits for the patient. In another embodiment, the practitioner can use a stimulation system 420 having an array of electrodes 422 when it is initially uncertain which area(s) of the patient's brain 150 should be stimulated to produce the most beneficial effect. Accordingly, the practitioner can stimulate a particular area of the brain 150 with one or more of the electrodes 422, observe the effect on the patient and, if the effect is not the desired effect, stimulate another area of the brain 150 with another of the electrodes 422 and observe the resulting effect, all with a single implanted stimulation system 420.

In still another embodiment, the practitioner can apply stimulation to different sites for different lengths of time, and/or the practitioner can independently vary other stimulation parameters supplied to the electrodes 422. In any of these embodiments, any characteristic or combination of characteristics of the signal applied to the electrodes 422 can be varied randomly, pseudo-randomly, aperiodically or approximately aperiodically. Further details of the signals applied to the electrodes 422 are described below with reference to FIG. 5.

FIG. 5 illustrates an electrode system 520 generally similar to that described above with reference to FIG. 4 and having a support 521 carrying a plurality of electrodes 522 arranged along a single line. One or more of the electrodes 522 can be positioned at least proximate to implanted cells. In other embodiments, the electrodes 522 can be arranged along multiple axes (as shown in FIG. 4), or in an irregular pattern. The system 520 can also include a pulse generator 540. For purposes of illustration, two alternative examples of pulse generators 540 are shown in FIG. 5 as a first pulse generator 540a and a second pulse generator 540b. The first pulse generator 540a can be implanted at a subclavicular location in the patient P, and the second pulse generator 540b can be implanted above the neck, posteriorly to the ear of the patient P. Either pulse generator 540 can be coupled to the electrode support 521 with a lead 541 and can provide electrical signals that stimulate the adjacent cells, as described in greater detail below.

In one embodiment, the electrical signals can be applied to a single one of the electrodes 522 to provide a unipolar pulse of current to a small area of the brain 150. Accordingly, the system 520 can include a return electrode, which can be a portion of the pulse generator 540, or a separate electrode implanted elsewhere in the patient P (e.g., on the other side of the patient's brain 150 or at a subclavicular location). In other embodiments, electrical current can be passed through all of the electrodes 522 or only a subset of the electrodes 522 to stimulate larger or different populations of implanted cells and/or native neurons. In one aspect of these embodiments, the potential applied to the electrodes 522 can be the same across all of the activated electrodes 522 to provide unipolar stimulation at the stimulation site. In other embodiments, some of the electrodes 522 can be biased with a positive polarity and other electrodes 522 can be biased with a negative polarity at any given point in time. This embodiment provides a bipolar stimulation to the brain 150. The particular configuration of the electrodes 522 activated during treatment can be optimized after implantation to provide the most efficacious therapy for the patient P.

The particular waveform of the applied stimulus depends upon the symptoms of the patient P. In one embodiment, the stimulus includes a series of biphasic, charge balanced pulses. In one aspect of this embodiment, each phase of the pulse is generally square. In another embodiment, the first phase can include a generally square wave portion representing an increase in current above a reference level, and a decrease below the reference level. The second phase can include a gradual rise back to the reference level. The first phase can have a pulse width ranging from about 25 microseconds to about 400 microseconds. In particular embodiments, the first phase can have a pulse width of 100 microseconds or 250 microseconds. The total pulse width can range up to 500 milliseconds.

The voltage of the stimulus can have a value of from about 0.25 V to about 10.0 V. In further particular embodiments, the voltage can have a value of from about 0.25 V to about 5.0 V, about 0.5 V to about 3.5 V, about 2.0 V to about 3.5 V or about 3 V. The voltage can be selected to correspond in some manner to the target or actual action potential of the stimulated cells. For example, if the stimulated cells have differentiated to the point that they exhibit action potentials, the applied voltage can be correlated with the exhibited threshold potential. If the stimulated cells do not yet exhibit action potentials, the applied voltage can be correlated to the desired threshold potential. In either embodiment, the selected voltage can be below a level that causes movement, speech or sensation in the patient (e.g., subthreshold) or above such a level (e.g., suprathreshold). Accordingly, the threshold level is generally correlated with generating electrophysiologic signals associated with a neural function. In one embodiment, the subthreshold voltage can be from about 10% to about 50% less than the threshold voltage. In another embodiment, the subthreshold voltage can have other ranges, for example, from about 10% to about 95%, about 10% to about 60%, about 20% to about 60%, about 30% to about 60%, about 25% to about 50%, about 60% to about 80%, or about 50% to about 80% less than the threshold voltage. In certain embodiments, the practitioner may control the current applied to the patient, in addition to or in lieu of controlling the voltage applied to the patient. Once the implanted cells begin to exhibit action potentials, the voltage and/or current applied to the cells can be reduced, or in further particular embodiments, the electrical stimulation can cease.

In particular embodiments, the voltage of the stimulus (or any other characteristic of the stimulus) is adjusted and/or selected based on a response by and/or characteristic of the implanted cells. Techniques (e.g., fMRI techniques) can be used to isolate the response and/or characteristic as being attributed to the implanted cells. In other embodiments, the response and/or characteristic may be attributed to native cells and this attribution can be made on the basis of similar techniques. In still further embodiments, the response and/or characteristic may be attributed to both native cells and implanted cells. In yet further embodiments, it is not necessary to attribute the response and/or characteristic to a particular type of cell (e.g., native cell and/or implanted cell). The implanted cells may develop functionality via excitatory and/or inhibitory pathways. Native cells, though possibly damaged, may influence the functionality (e.g., the ability to generate action potentials) of the implanted cells. In any of the foregoing embodiments, the response and/or characteristic of the cells can be determined on the basis of the cells' action potentials (or lack of action potentials) or by other techniques, for example, specific and/or general aspects of the patient's response to the stimulation.

The frequency of the stimulus can have a value of from about 2 Hz to about 250 Hz. In particular embodiments, the frequency can have a value of from about 50 Hz to about 150 Hz, or about 100 Hz. The stimulation can be applied for a period of 0.5 hour-4.0 hours, and in many applications the stimulation can be applied for a period of approximately 0.5 hour-2.0 hours, either during therapy (e.g., physical therapy or language comprehension training) or before, during and/or after such therapy. In other embodiments, the stimulation can be applied continuously, or only during waking periods but not during sleeping periods. In particular aspects of this embodiment, the characteristics (e.g., current, voltage, waveform, pulse duration, frequency) are different depending on whether the stimulation is applied before, during or after the therapy. In still further embodiments, the stimulation can be applied while a selected drug (e.g., an amphetamine or other neuroexcitatory agent) is active. In other embodiments, such drugs are not administered. Examples of specific electrical stimulation protocols for use with an electrode array at an epidural stimulation site are as follows:

### EXAMPLE 1

An electrical stimulus having a current of from about 3 mA to about 10 mA, an impedance of 500 to 2000 Ohms, a pulse duration of 160 microseconds, and a frequency of approximately 100 Hz. The therapy is not applied continuously, but rather during 30-120 minute intervals, associated with therapy.

### EXAMPLE 2

The stimulus has a current of from about 3 mA to about 6 mA, a pulse duration of approximately 150-180 microseconds, and a frequency of approximately 25 Hz-31 Hz. The stimulus is applied continuously during waking periods, but it is discontinued during sleeping periods to conserve battery life of the implanted pulse generator.

### EXAMPLE 3

The stimulus has a current of from about 3 mA to about 6 mA, a pulse duration of approximately 90 microseconds, and a frequency of approximately 30 Hz. This stimulus is applied continuously during waking and sleeping periods, but it can be selectively discontinued during sleeping periods.

Treatment programs in accordance with several embodiments of the invention can include electrical stimulation by itself, and/or electrical stimulation in conjunction or association with one or more synergistic or adjunctive therapies, such as behavioral therapies, activities, and/or tasks. Such behavioral therapies, activities, and/or tasks can include physical therapy; physical and/or cognitive skills training or practice, such as training in Activities of Daily Living (ADL); intentional use of an affected body part; speech therapy; vision training or visual tasks; a reading task; a memory task or memory training; comprehension tasks; attention tasks; an imagination or visualization task; and/or other therapies or activities. Other synergistic or adjunctive therapies can include, for example, drug therapies, such as treatment with amphetamines. The electrical stimulation and synergistic or adjunctive therapies can be performed simultaneously and/or serially.

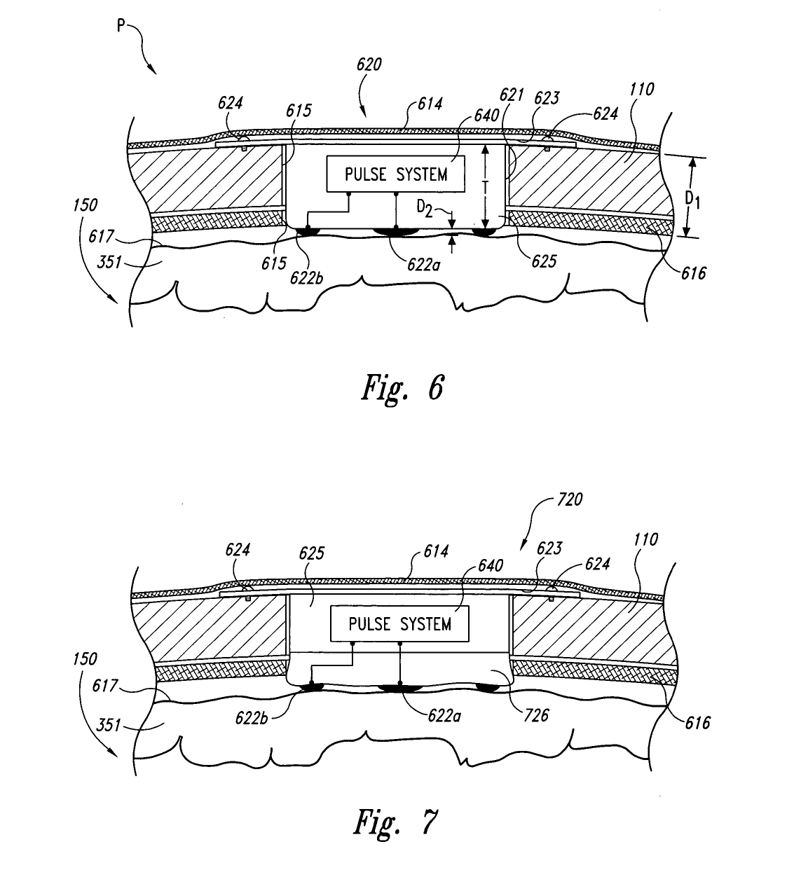

In one aspect of embodiments of the systems described above with reference to FIGS. 4 and 5, an electrode assembly having multiple electrodes is positioned at the cortex of the brain 150. Further details of such placements are described below with reference to FIGS. 6-8. In other embodiments, portions of the electrode assemblies can extend into or beneath the cortex to stimulate interior portions of the brain 150, including deep brain tissue (e.g., the substantia nigra 364 shown in FIG. 3). Electrode assemblies having suitable configurations for stimulating cells at these locations are available from Medtronic, Inc. of Minneapolis, Minn. In another embodiment, cells in the interior portions of the brain 150 can be stimulated from cortically positioned electrodes via the intermediate tissue. In still further embodiments, the electrode assembly can include a single electrode or one or more electrode pairs, also described in greater detail below with reference to FIGS. 6-8.

FIG. 6 is a cross-sectional view of a stimulation system 620 configured and implanted in accordance with an embodiment of the invention. In one aspect of this embodiment, the stimulation system 620 includes a support member 621, an integrated pulse system 640 (shown schematically) carried by the support member 621, and first and second electrodes or contacts 622 (identified individually by reference numbers 622a and 622b). The first and second electrodes 622 are electrically coupled to the pulse system 640 and are carried by the support member 621.

The support member 621 can be configured to be implanted in the skull 110 or another region of a patient P above the neckline. In one embodiment, for example, the support member 621 includes a housing 625 and an attachment element 623 connected to the housing 625. The housing 625 can be a molded casing formed from a biocompatible material, and can have an interior cavity for carrying the pulse system 640 and a power supply. The housing 625 can alternatively be a biocompatible metal or another suitable material. The housing 625 can have a diameter of approximately 1-4 cm, and in many applications the housing 625 can be 1.5-2.5 cm in diameter. The thickness T of the housing 625 can be approximately 0.5-4 cm, and can more generally be about 1-2 cm. The housing 625 can also have other shapes (e.g., rectilinear, oval, elliptical) and/or other surface dimensions. The stimulation system 620 can weigh 35 g or less and/or can occupy a volume of 20 cc or less. The attachment element 623 can include a flexible cover, a rigid plate, a contoured cap, or another suitable element for holding the support member 621 relative to the skull 110 or other body part of the patient P. In one embodiment, the attachment element 623 includes a mesh, e.g., a biocompatible polymeric mesh, metal mesh, or other suitable woven material. The attachment element 623 can alternatively be a flexible sheet of Mylar, polyester, or another suitable material.

In one aspect of an embodiment shown in FIG. 6, the stimulation system 620 is implanted in the patient P by forming an opening in the scalp 614 and cutting a hole 615 completely through the skull 110. The hole 615 can also pass through the dura mater 616 for subdural applications (shown), or the hole 615 can pass through the skull 110 but not the dura mater 616 for epidural applications. The hole 615 can be sized to receive the housing 625 of the support member 621, and in most applications the hole 615 can be smaller than the attachment element 623. A practitioner can insert the support member 621 into the hole 615 and then secure the attachment element 623 to the skull 110. The attachment element 623 can be secured to the skull 110 using a plurality of fasteners 624 (e.g., screws, spikes, etc.) or an adhesive. In another embodiment, a plurality of downwardly depending spikes can be formed integrally with the attachment element 623 to provide anchors that can be driven into the skull 110.

The embodiment of the stimulation system 620 shown in FIG. 6 is configured to be implanted in the patient P so that the electrodes 622 are juxtaposed to a desired cortical stimulation site. The housing 625 can project from the attachment element 623 by a distance D, such that the electrodes 622 are positioned at least proximate to the dura mater 616 or the pia mater 617 surrounding the cortex 351. The electrodes 622 can project from the housing 625 as shown in FIG. 6. In the particular embodiment shown in FIG. 6, the electrodes 622 project from the housing 625 by a distance D₂ so that the electrodes 622 press against a desired surface of the brain 150. The distance D₂ is from 0.1 mm to about 5 cm in some embodiments, and has other values in other embodiments. In still further embodiments, the electrodes 622 are flush with the housing 625. The electrodes 622 can be separate conductive members attached to the housing 625, or the electrodes 622 can be integral surface regions of the housing 625.

The configuration of the stimulation system 620 is not limited to the embodiment shown in FIG. 6. For example, in other embodiments, the housing 625, and the attachment element 623 can be configured to position the electrodes 622 in several different regions of the brain. In particular embodiments, the housing 625 and the attachment element 623 can be configured to position the electrodes 622 deep within the cortex 351 or against the dura mater 616.

The pulse system 640 shown in FIG. 6 generates and/or transmits electrical pulses to the electrodes 622 to stimulate a cortical region of the brain 150. The particular embodiment of the pulse system 640 shown in FIG. 6 is an “integrated” unit in that the pulse system 640 is carried by the support member 621. The pulse system 640, for example, can be positioned within the housing 625 so that the electrodes 622 can be carried by the housing 625 and connected directly to the pulse system 640 without having external leads outside the stimulation system 620. The distance between the electrodes 622 and the pulse system 640 can be less than 4 cm, for example, 0.10 to 2.0 cm. The stimulation system 620 can accordingly provide electrical pulses to the stimulation site without requiring a remote implanted pulse generator, which is connected to the electrodes 622 with surgically tunneled cables. In other embodiments, the pulse generator can be implanted separately from the electrodes, for example, in a manner generally similar to that described above with reference to FIG. 5. In still further embodiments, signals can be transmitted to the electrodes 622 from a remote location outside the patient's body via a wireless (e.g., RF) link.

FIG. 7 is a cross-sectional view of a stimulation system 720 configured and implanted in accordance with another embodiment of the invention. In one aspect of this embodiment, the stimulation system 720 includes a driving element 726 coupled to the electrodes 622 to mechanically urge the electrodes 622 away from the housing 625. In another embodiment, the driving element 726 can be positioned between the housing 625 and the attachment element 623, and the electrodes 622 can be attached directly to the housing 625. The driving element 726 can include a compressible member, for example, an open or closed cell biocompatible compressible foam, or a compressible solid (e.g., silicon rubber). In other embodiments, the driving element 726 can include a fluid-filled bladder, a spring, or any other suitable element that resiliently and/or elastically exerts a force against the electrodes 622.

In one aspect of an embodiment shown in FIG. 7, the driving element 726 is compressed slightly upon implantation so that the electrodes 622 contact the stimulation site. For example, the compressed driving element 726 can gently press the electrodes 622 against the surface of the pia mater 617. It is expected that the driving element 726 will provide a uniform, consistent contact between the electrodes 622 and a stimulation site surface, e.g., the pial or dural surface of the cortex 351. The stimulation system 720 is expected to be particularly useful when the implantable device is attached to the skull 110 and the stimulation site is on the pia mater 617 or the dura mater 616. It can be difficult to position the electrodes 622 against the pia mater 617 because the distance between the skull 110 and the dura mater 616 or the pia mater 617 varies as the brain 150 expands and contracts relative to the skull 110, and also because this distance varies from one patient P to another. The driving element 726 of the stimulation system 720 can compensate for the different distances between the skull 110 and the pia mater 617 so that a single type of device can better fit several different patients P. Moreover, the driving element 726 can change the position of the electrodes 622 as the brain 150 moves within the skull 110.

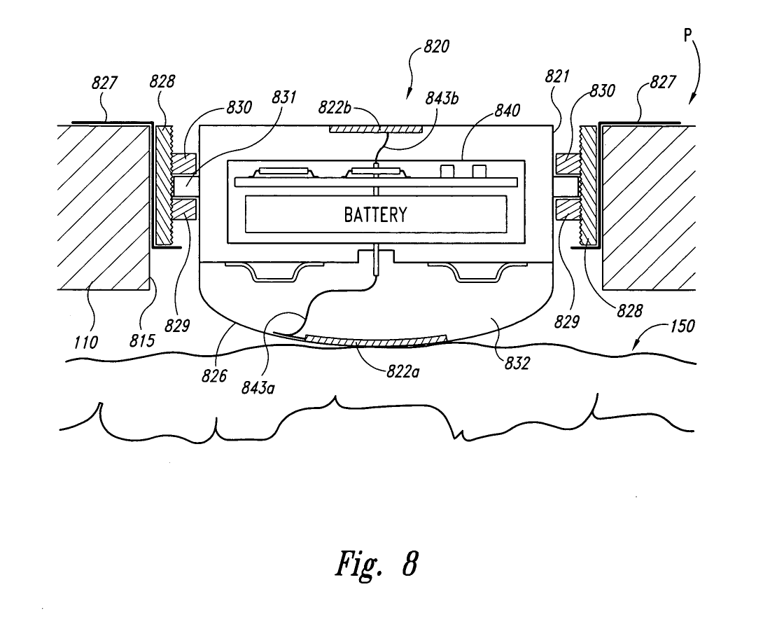

FIG. 8 is a cross-sectional view of a stimulation system 820 configured and implanted in accordance with another embodiment of the invention. The stimulation system 820 can include a support member 821, an integrated pulse system 840 (shown schematically) carried by the support member 821, a driving element 826 carried by the support member 821, and an electrode or contact 822a carried by the driving element 826. The contact 822a is electrically coupled to the pulse system 840 by a lead 843a. The driving element 826 can be a compliant material having a cavity 832 filled with a fluid such as saline or air. In another embodiment, the stimulation system 820 can further include an optional return electrode 822b carried on the opposite side of the support structure 821. The return electrode 822b can be electrically coupled to the pulse system 840 by a return lead 843b.

To implant the stimulation apparatus 820, a burr hole 815 is cut completely through the skull 110 of the patient P at a predetermined location identified according to the methods set forth above. The burr hole 815 can also pass through the dura mater (not shown FIG. 8). After forming the burr hole 815, a ferrule 827 is placed in the burr hole 815, and a threaded barrel 828 is welded or otherwise attached to the ferrule 827. A position ring 829 is then threaded along the threads of the barrel 828 to a desired height. The stimulation system 820 is placed in the burr hole 815 until a rim 831 projecting from the support member 821 engages the position ring 829. A lock ring 830 is then threaded onto the barrel 829 until it engages the rim 831. The position ring 829 and the lock ring 830 hold the support member 821 at a desired height relative to the surface of the patient's brain 150.

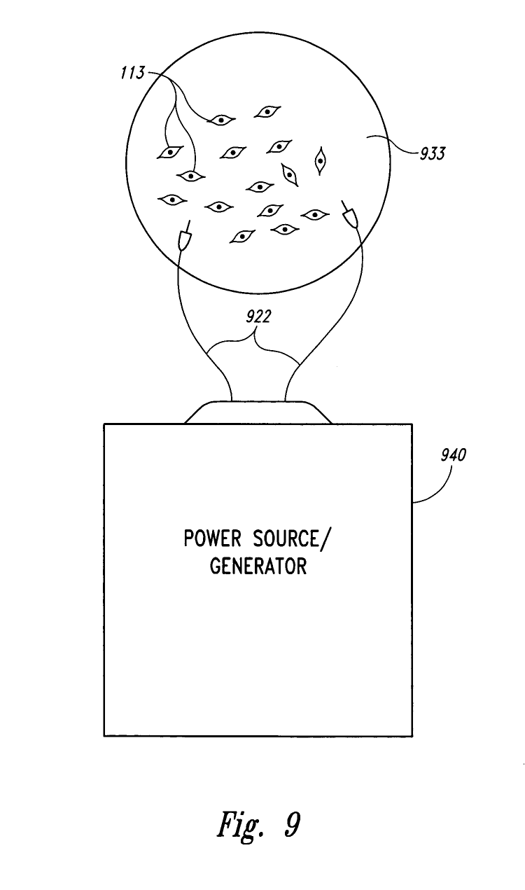

FIG. 9 schematically illustrates a procedure for electrically stimulating cell 113 in accordance with another embodiment to the invention. In one aspect of this embodiment, the cells 113 are grown in a conductive medium 933. A pulse generator 940 can deliver an electrical current in vitro to electrodes 922 (or electrode plates) implanted in the conductive medium 933 to electrically stimulate the cells 113. After the cells 113 have been electrically stimulated for a selected period of time, they can be removed from the conductive medium 933 and then implanted in the patient P as described above with reference to FIG. 1B. For example, the cells 113 can be stimulated in vitro until they begin to exhibit characteristics of action potential cells. As described above, the electrical stimulation can be continued after implantation with the same or different current voltage and frequency characteristics.

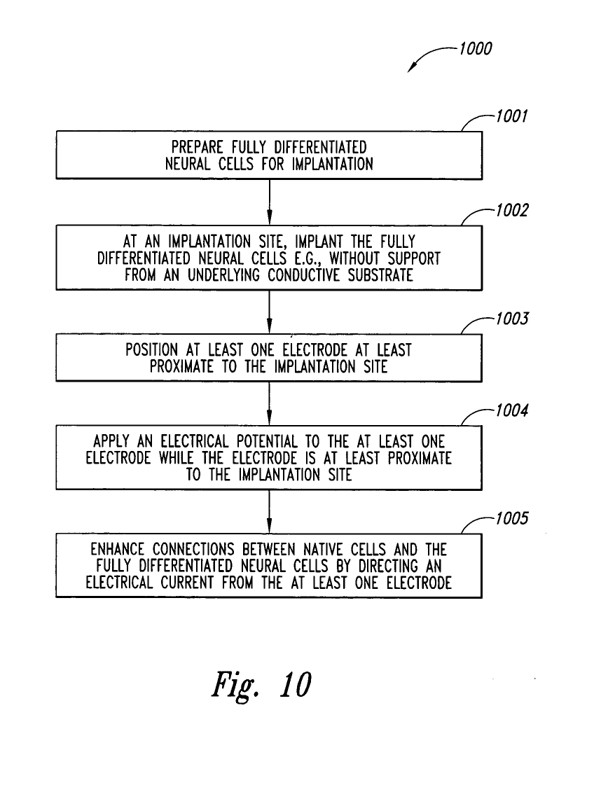

Still further embodiments of the invention use electrical stimulation in conjunction with fully differentiated implanted cells. For example, referring first to FIG. 10, a method 1000 in accordance with one aspect of the invention includes preparing fully differentiated neural cells for implantation, and implanting the cells at an implantation site of the patient's brain. At least one electrode is positioned at least proximate to the implantation site and an electrical potential is applied to the at least one electrode. The method can further include enhancing connections between native cells and the fully differentiated neural cells by directing an electrical current from the at least one electrode through the tissue surrounding the fully differentiated neural cells.

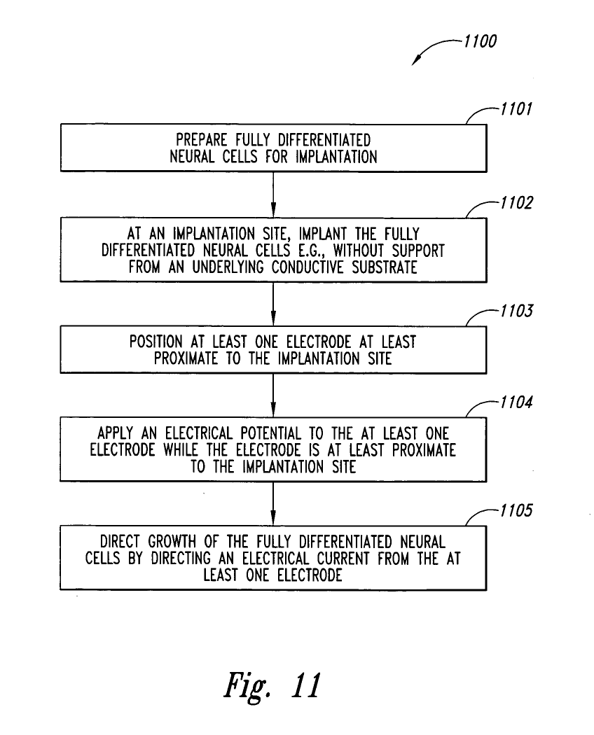

In another embodiment, shown in a flow diagram in FIG. 11, the growth of fully differentiated neural cells can be directed with an electrical stimulus. Accordingly, a process 1100 in accordance with one embodiment includes preparing fully differentiated neural cells for implantation (process portion 1101) and implanting the cells directly into the patient's brain. In a particular aspect of this embodiment, the cells are implanted directly into the brain tissue without an underlying conductive substrate. At least one electrode is positioned at least proximate to the implantation site (process portion 1102) and an electrical potential is applied to the at least one electrode (process portion 1103). The growth of the fully differentiated neural cells is directed by directing an electrical current from the at least one electrode through the tissue surrounding the fully differentiated neural cells. Advantages of embodiments of the methods described above with reference to FIGS. 10 and 11 are that the growth of fully differentiated neural cells and/or the connections between such cells and adjacent cells can be enhanced with electrical current, but in a particular embodiment without requiring a conductive substrate attached to the implanted cells.

In a further particular embodiment, the growth of the fully differentiated, implanted neural cells can be directed by applying electrical current from a device having a plurality of electrodes, for example, a device generally similar to the stimulation system 520 described above with reference to FIG. 5. Returning now to FIG. 5, the support 521 can be implanted so that the electrodes 522 are aligned with a target direction of growth for the fully differentiated neural cells. In one aspect of this embodiment, electrodes 522 arranged sequentially along the path can receive sequential electrical impulses to encourage the neural cells to grow along the target path. For example, one electrode 522 can receive signals until the cell grows proximate to that electrode, at which point, the next electrode 522 along the path can receive stimulation signals. In another embodiment, more than one of the electrodes 522 can receive electrical signals simultaneously. In still a further aspect of this embodiment, the nature of the electrical signals directed to sequentially located electrodes 522 can be different. For example, electrodes located at different positions along the target path can receive electrical signals having different voltages, currents, and/or frequency modulations. In still further embodiments, any of the foregoing techniques can be applied to directing the growth of at least unpartially undifferentiated cells, in addition to or in lieu of the fully differentiated cells described above.

From the foregoing, it will be appreciated that specific embodiments of the invention have been described herein for purposes of illustration, but that various modifications may be made without deviating from the spirit and scope of the invention. Accordingly, the invention is not limited except as by the appended claims.

1. A method for treating a neural disorder, comprising:
preparing cells for implantation, the cells being in a first, at least partially undifferentiated state;
implanting the cells at an implantation site within the skull cavity of a patient while the cells are in the first state;
positioning at least one electrode in electrical communication with the implantation site of the patient; and
at least partially correcting a neural dysfunction at least proximate to the implantation site by differentiating the cells at least until the cells achieve a second state, the cells in the second state having an increased level of differentiation and increased neural characteristics when compared to the cells in the first state, wherein differentiating the cells includes applying an electrical potential to the at least one electrode while the electrode is in electrical communication with the implantation site of the patient.

2. The method of claim 1 wherein implanting the cells includes implanting the cells directly into tissue of the patient without the cells being carried by an electrically conductive substrate.

3. The method of claim 1, further comprising removing the at least one electrode from the patient without removing the implanted cells.

4. The method of claim 1 wherein implanting the cells includes implanting the cells at at least one of an infarct region and a peri-infarct region of the nervous system.

5. The method of claim 1 wherein implanting the cells includes implanting the cells at least proximate to the basal ganglia of the patient.

6. The method of claim 1 wherein implanting the cells includes implanting the cells at least proximate to the motor cortex of the patient.

7. The method of claim 1 wherein implanting the cells includes implanting the cells at least proximate to the thalamus of the patient.

8. The method of claim 1 wherein implanting the cells includes implanting the cells at least proximate to the ventralis intermedius nucleus of the thalamus of the patient.

9. The method of claim 1 wherein implanting the cells includes implanting the cells at least proximate to the putamen of the patient.

10. The method of claim 1 wherein implanting the cells includes implanting the cells at least proximate to the globus pallidus of the patient.

11. The method of claim 1 wherein implanting the cells includes implanting the cells at least proximate to the subthalamic nucleus of the patient.

12. The method of claim 1 wherein implanting the cells includes implanting the cells at least proximate to at least one of Broca's area, Wernicke's area, and neuronal connections extending between Broca's area and Wernicke's area.

13. The method of claim 1 wherein differentiating the cells includes at least partially reversing neural damage resulting from Huntington's disease.

14. The method of claim 1 wherein differentiating the cells includes at least partially alleviating essential tremor motion.

15. The method of claim 1 wherein differentiating the cells includes at least partially alleviating a movement disorder.

16. The method of claim 1 wherein differentiating the cells includes at least partially reversing neural damage resulting from Parkinson's disease.

17. The method of claim 1 wherein differentiating the cells includes at least partially reversing neural damage resulting from a stroke.

18. The method of claim 1, further comprising exposing the cells to growth factors.

19. The method of claim 1, further comprising exposing the cells to at least one of IGF and GDNF.

20. The method of claim 1 wherein applying an electrical potential to the at least one electrode includes applying the electrical potential before implanting the cells.

21. The method of claim 1 wherein applying an electrical potential to the at least one electrode includes applying the electrical potential after implanting the cells.

22. The method of claim 1, further comprising transporting a growth factor into the cells with a virus.

23. The method of claim 1 wherein positioning at least one electrode includes positioning at least one electrode proximate to a native cell and communicating with the implanted cells via the native cell.

24. The method of claim 1 wherein the cells are selected to include stem cells, precursor cells, and/or progenitor cells.

25. The method of claim 1 wherein the at least one electrode includes a first electrode, and wherein the method further comprises positioning a second electrode at least proximate to the implantation site, and wherein applying an electrical potential includes applying a voltage of from about ±0.25V to about ±10 V between the first electrode and the second electrode while the electrodes are at least proximate to the implantation site.

26. The method of claim 1 wherein applying an electrical potential includes generating electrical pulses at a rate of from about 2 to about 250 Hz.

27. The method of claim 1 wherein applying an electrical potential includes applying a current of from about 3 mA to about 10 mA.

28. The method of claim 1 wherein differentiating the cells includes applying an electrical potential to the at least one electrode at a first voltage until the cells develop action potentials and then applying an electrical potential to the at least one electrode at a second voltage less than the first voltage after the cells develop action potentials.

29. The method of claim 1 wherein differentiating the cells includes ceasing to apply an electrical potential to the at least one electrode after the cells develop increased action potentials.

30. The method of claim 1, further comprising ascertaining a threshold for generating action potentials for the cells at the implantation site, and wherein applying an electrical potential includes applying a subthreshold voltage less than the threshold for generating action potentials.

31. The method of claim 1 wherein the at least one electrode includes a first electrode, and wherein the method further comprises:
ascertaining a threshold for generating action potentials for the cells at the implantation site; and
positioning a second electrode at least proximate to the implantation site, and wherein applying an electrical potential includes placing a subthreshold voltage between the first electrode and the second electrode, wherein the subthreshold voltage is approximately 10% to approximately 50% less than the threshold for generating an action potential.

32. The method of claim 1 wherein the at least one electrode includes a first electrode, and wherein the method further comprises:
ascertaining a threshold for generating electrophysiologic signals associated with a neural function; and
positioning a second electrode at least proximate to the implantation site of the nervous system, and wherein applying an electrical potential includes placing a subthreshold voltage between the first electrode and the second electrode, wherein the subthreshold voltage is less than the threshold for generating electrophysiologic signals.

33. The method of claim 1 wherein the at least one electrode includes a first electrode, and wherein the method further comprises:
ascertaining a threshold for generating electrophysiologic signals for the cells at the implantation site; and
positioning a second electrode at least proximate to the implantation site, wherein applying an electrical potential includes applying a subthreshold voltage between the first electrode and the second electrode, wherein the subthreshold voltage is from about 20% to about 50% less than the threshold for generating electrophysiologic signals.

34. The method of claim 1 wherein the at least one electrode includes a first electrode, and wherein the method further comprises:
ascertaining a threshold for eliciting a neural function; and
positioning a second electrode at least proximate to the implantation site, and wherein applying an electrical potential includes placing a subthreshold voltage between the first electrode and the second electrode, wherein the subthreshold voltage is less than the threshold for eliciting the neural function.

35. The method of claim 1 wherein the at least one electrode includes a first electrode, and wherein the method further comprises:
ascertaining a threshold for eliciting a neural function; and
positioning a second electrode at least proximate to the implantation site, and wherein applying an electrical potential includes placing a subthreshold voltage between the first electrode and the second electrode, wherein the subthreshold voltage is from about 30% to about 60% less than the threshold for eliciting the neural function.

36. The method of claim 1, further comprising identifying a stimulation site by generating remotely from the stimulation site an intended neural activity and determining the location of the brain where the generated neural activity is present.

37. The method of claim 1, further comprising implanting a pulse generator at least proximate to the implanted cells.

38. A method for treating a neural disorder, comprising:
identifying a site of the brain for implantation and stimulation;
preparing cells for implantation, the cells being in a first, at least partially undifferentiated state;
implanting the cells at the site while the cells are in the first state, the cells being unsupported by an electrically conductive substrate;
positioning at least one electrode in electrical communication with the site via native cells; and
at least partially correcting a neural dysfunction at least proximate to the implantation site by differentiating the cells at least until the cells achieve a second state, the cells in the second state having an increased level of differentiation and increased neural characteristics when compared to the cells in the first state, wherein differentiating the cells includes applying an electrical potential to the at least one electrode while the electrode is in electrical communication with the implantation site of the patient.

39. The method of claim 38, further comprising removing the at least one electrode from the patient without removing the implanted cells.

40. The method of claim 38, further comprising implanting a pulse generator at least proximate to the implanted cells.

41. The method of claim 38 wherein identifying the site includes stimulating a peripheral nerve of the patient and obtaining information corresponding to simultaneous activity in the patient's brain.

42. The method of claim 38 wherein identifying the site includes directing the patient to engage in a language-based task and obtaining information corresponding to simultaneous activity in the patient's brain.

43. A method for treating a neural disorder, comprising:
preparing fully differentiated neural cells for implantation;
implanting the cells at an implantation site within the skull cavity of a patient;
positioning at least one electrode at least proximate to the implantation site;
applying an electrical potential to the at least one electrode while the electrode is at least proximate to the implantation site of the nervous system; and
enhancing connections between native cells and the fully differentiated neural cells by directing an electrical current from the at least one electrode through the tissue surrounding the fully differentiated neural cells.

44. The method of claim 43 wherein implanting the fully differentiated neural cells includes implanting the cells directly into tissue of the patient without the cells being carried by an electrically conductive substrate.

45. The method of claim 43 wherein preparing fully differentiated neural cells includes applying an electrical stimulation to the cells while the cells are external to a patient.

46. The method of claim 43 wherein positioning at least one electrode includes positioning at least one electrode proximate to a native cell and communicating with the implanted cells via the native cell.

47. The method of claim 43 wherein the at least one electrode includes a first electrode, and wherein the method further comprises positioning a second electrode at least proximate to the implantation site, and wherein applying an electrical potential includes applying a voltage of from about ±0.25V to about ±10 V between the first electrode and the second electrode while the electrodes are at least proximate to the implantation site.

48. The method of claim 43 wherein applying an electrical potential includes generating electrical pulses at a rate of from about 2 to about 250 Hz.

49. The method of claim 43 wherein applying an electrical potential includes applying a current of from about 3 mA to about 10 mA.

50. The method of claim 43, further comprising identifying a stimulation site by generating remotely from the stimulation site an intended neural activity and determining the location of the brain where the generated neural activity is present.

51. The method of claim 43, further comprising implanting a pulse generator at least proximate to the implanted cells.

52. The method of claim 43, further comprising identifying the implantation site.

53. A method for treating a neural disorder, comprising:
preparing fully differentiated neural cells for implantation;
implanting the cells at an implantation site within the skull cavity of a patient;
positioning at least one electrode at least proximate to the implantation site;
applying an electrical potential to the at least one electrode while the electrode is at least proximate to the implantation site of the nervous system; and
directing growth of the cells by directing an electrical current from the at least one electrode through the tissue surrounding the fully differentiated neural cells.

54. The method of claim 53 wherein positioning at least one electrode includes implanting a plurality of electrodes along a growth path, and wherein the method further comprises applying electrical potentials to the electrodes in a sequential manner along the growth path to direct the growth of the cells along the growth path.

55. The method of claim 53 wherein implanting the fully differentiated neural cells includes implanting the cells directly into tissue of the patient without the cells being carried by an electrically conductive substrate.

56. The method of claim 53 wherein preparing fully differentiated neural cells includes applying an electrical stimulation to the cells while the cells are external to a patient.

57. The method of claim 53 wherein positioning at least one electrode includes positioning at least one electrode proximate to a native cell and communicating with the implanted cells via the native cell.

58. The method of claim 53 wherein the at least one electrode includes a first electrode, and wherein the method further comprises positioning a second electrode at least proximate to the implantation site, and wherein applying an electrical potential includes applying a voltage of from about ±0.25V to about ±10 V between the first electrode and the second electrode while the electrodes are at least proximate to the implantation site.

59. The method of claim 53 wherein applying an electrical potential includes generating electrical pulses at a rate of from about 2 to about 250 Hz.

60. The method of claim 53 wherein applying an electrical potential includes applying a current of from about 3 mA to about 10 mA.

61. The method of claim 53, further comprising identifying a stimulation site by generating remotely from the stimulation site an intended neural activity and determining the location of the brain where the generated neural activity is present.

62. The method of claim 53, further comprising implanting a pulse generator at least proximate to the implanted cells.

63. The method of claim 53, further comprising identifying the implantation site.
